(19) United States
(12) Patent Application Publication
(10) Pub. No.: US 2017/0216186 A1
(43) Pub. Date: Aug. 3, 2017

(54) MEANS AND METHODS FOR TEMPORARILY DEFORMING KERATIN-CONTAINING FIBERS

(71) Applicant: Henkel AG & Co. KGaA, Dusseldorf (DE)

(72) Inventors: Julia Bibiane Lange, Bad Bramstedt (DE); Anna Henschel Puls, Winsen (Luhe) (DE)

(73) Assignee: Henkel AG & Co. KGaA, Dusseldorf (DE)

(21) Appl. No.: 15/492,358

(22) Filed: Apr. 20, 2017

Related U.S. Application Data
(63) Continuation of application No. PCT/EP2015/070437, filed on Sep. 8, 2015.

(30) Foreign Application Priority Data
Oct. 21, 2014 (DE) 102014221375.4

(51) Int. Cl. A61K 8/81; A61Q 5/06

(52) U.S. Cl. CPC A61K 8/8182; A61Q 5/06; A61K 8/8152; A61K 2800/594

(57) ABSTRACT

The present invention relates to cosmetic agents for temporarily deforming keratinous fibers comprising—relative to the total weight thereof—a) 0.1 to 2.0 w % of at least one hydrophobically modified (meth) acrylic acid copolymer and b) 0.2 to 12 wt % of at least one polymeric quaternary ammonium compound from the group of vinylpyrrolidone copolymers, to the use thereof, and to methods using said agents.

### FIELD OF THE INVENTION

The present invention generally relates to a cosmetic agent for hair strengthening or for temporarily deforming keratinous fibers, in particular, human hair, wherein the agent includes a combination of two polymers.

### BACKGROUND OF THE INVENTION

Temporary deforming of hairstyles for a longer period of time, up to several days, generally requires the use of fixing agents. Therefore, hair treatment agents that are used to temporarily give shape to the hair play an important role. Corresponding agents for temporary deforming typically include synthetic polymers and/or waxes as fixing agents. Agents for supporting the temporary deformation of keratin-containing fibers may be prepared, for example, as a hair spray, hair wax, hair gel, or hair mousse.

The most important property of an agent for temporarily deforming hair—hereinafter also called a styling agent—is giving the strongest possible hold to the treated fibers in the newly-modeled form, i.e., a form imparted to the hair. Other topics of discussion are strong hairstyle holding or a high degree of holding of the styling agent. The hairstyle hold is determined essentially by the type and amount of the fixing agent used, wherein, however, the other components of the styling agent may also have an effect.

In addition to a high degree of hold, styling agents need to fulfill a whole range of other requirements. These can be broadly divided into: properties on the hair; properties of the respective formulation, e.g., properties of the foam, gel, or sprayed aerosol; and properties that affect the handling of the styling agent, wherein the properties on the hair are of special importance. These include, in particular, moisture resistance, strength, low tackiness, and a balanced conditioning effect. Furthermore, a styling agent should universally applicable to all hair types, as much as possible, and should be mild on the hair and skin.

To meet the different requirements, a large number of synthetic polymers that are used in styling agents have already been developed as fixing agents. The polymers can be divided into cationic, anionic, nonionic, and amphoteric fixing polymers. Ideally, when applied to the hair, the polymers produce a polymer film that, on one hand, gives very strong hold to the hairstyle but, on the other hand, is sufficiently flexible to not break under stress. German patent application DE 102012214380 A1 discloses an exemplary styling agent having favorable hold properties and high humidity resistance due to a combination of a hydrophobically modified (meth) acrylic acid copolymer and a hydrophobically modified polysaccharide.

German patent application DE 102011089170 A1 describes hair cosmetics that are suitable for temporarily deforming hair and include, in addition to other components, at least one polymeric quaternary ammonium compound from the group consisting of vinylpyrrolidone copolymers.

### BRIEF SUMMARY OF THE INVENTION

The problem is solved by the combination of two specific polymers. A first subject matter of the present invention is therefore a cosmetic agent for temporarily deforming keratinous fibers, including—relative to the total weight thereof—
a) 0.1 to 2.0 wt % at least one hydrophobically modified (meth)acrylic acid copolymer; and
b) 0.2 to 12 wt % at least one polymeric quaternary ammonium compound from the group of vinylpyrrolidone copolymer.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention addressed the problem of providing further suitable polymer combinations that are characterized by favorable film-forming and/or fixing properties, possess a very high degree of hold without needing to thereby compromise flexibility and favorable moisture resistance—in particular, resistance to sweat and water—and are additionally suitable for the production of cosmetic compositions having stable viscosity and, preferably, stable transparency. In particular, currently-available styling agents still have room for improvement in that a favorable combination of stiffness and long-term hold is not always sufficiently ensured. The present invention therefore addresses the problem of providing such styling agents that—in addition to the aforementioned properties—yield, in particular, both favorable rigidity and favorable long-lasting hold.

A first essential component of the cosmetic agents according to the present invention is the hydrophobically modified (meth)acrylic acid copolymer a). Preferred copolymers a) have a thickening action. A proportion by weight of the copolymer a) to the total weight of the cosmetic agent of 0.2 to 1.5 wt %, preferably 0.3 to 1.0 wt % and, in particular, 0.4 to 0.6 wt % has proven especially advantageous for the cosmetic effect. As the hydrophobically modified (meth)acrylic acid copolymers a), it is preferable to use copolymers that come from
at least one monomer (a1) from the group consisting of unsaturated carboxylic acids and unsaturated carboxylic acid esters, and
at least one monomer (a2) from the group consisting of unsaturated hydrophobically modified monomers.

Preferred copolymers a) are based on at least one monomer (a1) from the group consisting of acrylic acid, methacrylic acid, C₁-C₆ alkyl acrylic acid esters, and C₁-C₆ alkyl methacrylic acid esters. The acrylic acid esters and methacrylic acid esters are preferably esters of the respective acids with non-tertiary alkylalcohols having alkyl moieties of 1 to 12 carbon atoms, in particular, 2 to 4 carbon atoms. Suitable examples of monomers include ethyl acrylate, ethyl methacrylate, propyl acrylate, propyl methacrylate, n-butyl acrylate, n-butyl methacrylate, isobutyl acrylate, 2-methylbutyl acrylate, 2-ethylhexyl acrylate, n-octyl acrylate, isooctyl acrylate, isooctyl methacrylate, isononyl acrylate, and isodecyl acrylate.

The group consisting of hydrophobically modified monomers (a2) refers to monomers that feature a hydrophobic substructure. Preferred monomers (a2) come from, in turn, the two following structural units:
an unsaturated acid, preferably acrylic acid, methacrylic acid, or itaconic acid; and
a C₈₋₄₀ alkyl chain, preferably a C₁₀₋₃₀ alkyl chain.
   These two substructures may also optionally be supplemented with a third structural unit from the group consisting of polyoxyalkylene groups, preferably polyethylene glycol groups, polypropylene gylcol groups, or polyethylene glycol/polypropylene glycol groups.

By way of example, C₁₀₋₃₀ alkyl acrylates, C₁₀₋₃₀ alkyl PEG acrylates, C₁₀₋₃₀ alkyl PEG methacrylates, or C₁₀₋₃₀ alkyl PEG itaconates are used as the monomer (a2). Preferred monomers (a2) are selected from C₁₀₋₃₀ alkyl acrylates, C₁₀₋₃₀ alkyl PEG 20-25 acrylates, C₁₀₋₃₀ alkyl PEG 20-25 methacrylates, or C₁₀₋₃₀ alkyl PEG 20-25-itaconates. Especially preferred monomers (a2) are selected from the group consisting of C₁₀₋₃₀ alkyl acrylates, steareth-20 methacrylates, beheneth-25 methacrylates, steareth-20 itaconates, ceteth-20 itaconates, palmeth-25 acrylates, or C₁₀₋₃₀ alkyl PEG-20 itaconates.

In summary, preferred copolymers a) are formed from
at least one monomer (a1) from the group consisting of acrylic acid, methacrylic acid, C₁-C₆ alkyl acrylic acid esters, and C₁-C₆ alkyl methacrylic acid esters, with
at least one monomer (a2) from the group consisting of C₁₀₋₃₀ alkyl acrylates, C₁₀₋₃₀ alkyl PEG acrylates, C₁₀₋₃₀ alkyl PEG methacrylates, or C₁₀₋₃₀ alkyl PEG itaconates.

Especially preferred are copolymers a) that are obtained by reacting
at least one monomer (a1) from the group consisting of acrylic acid, methacrylic acid, C₁-C₆ alkyl acrylic acid esters, and C₁-C₆ alkyl methacrylic acid esters, with
at least one monomer (a2) from the group consisting of C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates, or C₁₀₋₃₀ alkyl PEG itaconates.

Further preferred hydrophobically modified (meth)acrylic acid copolymers a) are furthermore formed—beyond the aforementioned monomers (a1) and (a2)—from at least one monomers (a3) from the group consisting of unsaturated amine group-containing monomers.

As the monomer (a3), it is preferable to use the monomers from the group consisting of acrylamide, methacrylamide, mono-(C₁-C₄) alkylamino(C₁-C₄) alkyl acrylate, di-(C₁-C₄) alkylamino(C₁-C₄) alkyl acrylate, mono-(C₁-C₄) alkylamino(C₁-C₄) alkyl methacrylate, and di-(C₁-C₄) alkylamino(C₁-C₄) alkyl methacrylate. Exemplary and preferred monomers (A2) are 2-(N,N-dimethylamino)ethyl acrylate, 2-(N,N-dimethylamino)ethyl methacrylates, 2-(N,N-diethylamino)ethyl acrylates, 2-(N,N-diethylamino)ethyl methacrylates, 3-(N,N-dimethylamino)propyl acrylate, 3-(N,N-dimethylamino)propyl methacrylate, 2-(N,N-dimethylamino)neopentyl acrylate, N′-(3-N,N-dimethylamino)propyl acrylamide, and N′-(3-N,N-dimethylamino)propyl methacrylamide.

Preferred copolymers a) are formed from
at least one monomer (a1) from the group consisting of acrylic acid, methacrylic acid, C₁-C₆ alkyl acrylic acid esters, and C₁-C₆ alkyl methacrylic acid esters, with
at least one monomer (a2) from the group consisting of C₁₀₋₃₀ alkyl PEG 20 itaconates, and
at least one monomer (a3) from the group consisting of acrylamide, methacrylamide, mono-(C₁-C₄) alkylamino(C₁-C₄) alkyl acrylate, di-(C₁-C₄) alkylamino(C₁-C₄) alkyl acrylate, mono-(C₁-C₄) alkylamino(C₁-C₄) alkyl methacrylate, and di-(C₁-C₄) alkylamino(C₁-C₄) alkyl methacrylate.

It is preferable to use copolymers a) that are crosslinked. In summary, cosmetic agents that are preferred according to the present invention are characterized in that the copolymer a) is selected from the group consisting of compounds having the INCI designation Acrylates/C₁₀₋₃₀ alkyl Methacrylate Copolymer, Acrylates/C₁₀₋₃₀ alkyl Acrylate Crosspolymer, Acrylates/Steareth-20 Methacrylate Crosspolymer, Acrylates/Steareth-20 Methacrylate Copolymer, Acrylates/Beheneth-25 Methacrylate Copolymer, Acrylates/Steareth-20 Itaconate Copolymer, Acrylates/Ceteth-20 Itaconate Copolymer, Acrylates/Palmeth-25 Acrylate Copolymer, and Acrylates/Aminoacrylates/C₁₀₋₃₀ alkyl PEG-20 Itaconate Copolymer. Corresponding polymers are available, for example, under the tradenames Luvigel® FIT, Ultrez® 21, Pemulen® TR1, Aculyn® 22, Aculyn® 28, Aculyn® 88, Structure® 2001, Structure® 3001, Synthalen® W2000, and Structure® Plus. With particular preference, the copolymer a) is selected from the group consisting of compounds having the INCI designation Acrylates/C₁₀₋₃₀-Alkyl Methacrylate Crosspolymer.

As a second essential component, the agents according to the present invention include a polymeric quaternary ammonium compound from the group consisting of vinylpyrrolidone copolymers b). Cosmetic agents having, relative to the total weight of the cosmetic agent, a proportion by weight of the copolymer b) of 0.5 to 10 wt %, preferably 1.0 to 6.0 wt % and, in particular, 1.5 to 4.0 wt % are characterized by especially advantageous cosmetic properties.

Suitable copolymers b) are, for example:
quaternized vinylpyrrolidone/dimethylaminoethyl methacrylate copolymer (INCI designation: Polyquaternium 11), e.g., copolymers with the trade names Gafquat® 755 N and Gafquat® 734 (Gaf Co., USA) and Luviquat PQ 11 PN (BASF);
vinylpyrrolidone/imidazolimine methochloride copolymer (INCI designation: Polyquaternium 16), e.g., the copolymer Luviquat® HM 552 (BASF);
vinylpyrrolidone/methacrylamidopropyltrimethylammonium chloride copolymer (INCI designation: Polyquaternium 28), e.g., the copolymer Gafquat® HS 100 (ISP); methylvinylimidazolium chloride/vinylpyrrolidone copolymers (INCI designation: Polyquaternium 44), e.g., Luviquat® Ultra Care (BASF);
quaternized vinylpyrrolidone/vinyl caprolactam/vinylimidazole copolymer (INCI designation: Polyquaternium 46), e.g., Luviquat® Hold (BASF); and
quaternized vinylpyrrolidone/vinyl caprolactam/vinylimidazole/quaternized vinylimidazole copolymer (INCI designation: Polyquaternium 68), e.g., Luviquat® Supreme (BASF).

With particular preference, the agents according to the present invention include at least one copolymer selected from b1) copolymers of vinylpyrrolidone with methacrylamidopropyltrimethylammonium chloride (MAPTAC) and/or b2) copolymers of vinylpyrrolidone with dimethylaminoethyl methacrylate and/or b3) copolymers of vinylpyrrolidone with dimethylaminopropyl methacrylamide and alkyldimethylpropylmethacrylamidoammonium salts.

An especially suitable copolymer b) is obtained by reacting vinylpyrrolidone with methacrylamidopropyltrimethylammonium chloride.

These copolymers of vinylpyrrolidone with methacrylamidopropyltrimethylammonium chloride (MAPTAC) can be described by the formula

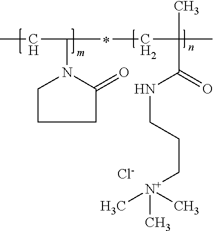

wherein the indices m and n vary depending on the molar mass of the polymer and should not signify that this is a block copolymer. Rather, structural units may be statistically distributed within the molecule.

Especially preferred cosmetic agents are characterized by including—as the cationic polymer b)—copolymers of methacrylamidopropyltrimethylammonium chloride (MAPTAC) with vinylpyrrolidone containing 40 to 95 mol %, preferably 42.5 to 90 mol %, further preferably 45 to 85 mol % and, in particular, 50 to 80 mol % vinylpyrrolidone.

Especially preferred cosmetic agents are further characterized in that the copolymers b) have a molar mass of 10 to 1,000 kDa, preferably 25 to 900 kDa, further preferably 50 to 800 kDa and, in particular, 100 to 750 kDa.

A very especially preferred copolymer b) is designated Polyquaternium 28 by the INCI nomenclature. Such a polymer is available, for example, under the tradename Gafquat® HS-100 (ISP).

In addition to the aforementioned advantages, the agents according to the present invention are also distinguished from cosmetic agents having alternative vinylpyrrolidone copolymers b) by an improved degree of hold and high stiffness. A weight ratio of 5:1 to 1:5, preferably 3:1 to 1:3 and, in particular, 2:1 to 1:2 of the polymers a) and b) in the cosmetic agent has proven especially advantageous for the cosmetic properties of the agents according to the present invention.

The copolymer a) is used preferably in a partially neutralized or neutralized form in the cosmetic agent. Preferably, at least one alkanolamine is used for neutralization. The alkanolamines that can be used as an alkalizing agent according to the present invention are preferably selected from primary amines having a C₂-C₆ alkyl parent substance bearing at least one hydroxy group. Especially preferred alkanolamines are selected from the group consisting of 2-aminoethan-1-ol (monoethanolamine), tris(2-hydroxyethyl) amine (triethanolamine), 3-aminopropan-1-ol, 4-aminobutan-1-ol, 5-aminopentan-1-ol, 1-aminopropane-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, and 2-amino-2-methylpropane-1,3-diol. Alkanolamines that are very especially preferred according to the present invention are selected from the group consisting of 2-aminoethan-1-ol, 2-amino-2-methylpropan-1-ol, and 2-amino-2-methylpropan-1,3-diol. 2-amino-2-methylpropanol has proven to be an especially suitable neutralizing agent. Cosmetic agents that are preferred according to the present invention include at least one alkanolamine, preferably 2-amino-2-methylpropanol. The 2-amino-2-methylpropanol is preferably used in the agents according to the present invention in an amount that does not exceed the amount required to neutralize the copolymer a). Preferably, the amount of 2-amino-2-methylpropanol used in the agents according to the present invention is 80 to 100%, especially preferably 90 to 100% and, in particular, 95% to 100% of the amount required to completely neutralize the copolymer a). In a preferred embodiment, the proportion by weight of the 2-amino-2-methylpropanol to the total weight of the cosmetic agent is 0.01 to 2.0 wt %, preferably 0.01 to 1.0 wt % and, in particular, 0.02 to 0.5 wt %. Very especially, the proportion by weight of the amino alcohol—preferably 2-amino-2-methylpropanol—is 0.01 to 0.2 wt %, preferably 0.01 to 0.1 wt % and, in particular, 0.02 to 0.6 wt %.

In addition to the aforementioned copolymers a) and copolymers b), the cosmetic agents according to the present invention may include additional active ingredients, auxiliary substances, and conditioners.

A first group of advantageously used active ingredients is film-forming polymers. These film-forming polymers are, here, not identical to the aforementioned hydrophobically modified (meth)acrylic acid copolymer a) nor to the vinylpyrrolidone copolymers b). The proportion by weight of the film-forming polymer to the total weight of the cosmetic agent is preferably 0.1 to 8.0 wt %, preferably 0.5 to 6.0 wt % and, in particular, 1.0 to 4.0 wt %.

Nonionic polymers are used with particular preference as the film-forming polymers. Examples of suitable nonionic polymers include:
Vinylpyrrolidone/vinyl ester copolymers, such as are marketed under the trademark Luviskol® (BASF). Luviskol® VA 64 and Luviskol® VA 73—each vinylpyrrolidone/vinyl acetate copolymers—are preferred nonionic polymers.
Cellulose ethers, such as hydroxypropyl cellulose, hydroxyethyl cellulose, and methylhydroxypropyl cellulose, such as are marketed, for example, under the trademarks Culminal® and Benecel® (AQUALON).
Shellac.
Polyvinylpyrrolidones, such as are marketed, for example, under the designation Luviskol® (BASF).
Siloxanes. These siloxanes may be both water-soluble and water-insoluble. Both volatile and non-volatile siloxanes are suitable, wherein non-volatile siloxanes are understood to be those compounds that have a boiling point at atmospheric pressure above 200° C. Preferred siloxanes are polydialkyl siloxanes, such as for example polydimethyl siloxane, polyalkylaryl siloxanes, such as for example polyphenylmethyl siloxane, ethoxylated polydialkyl siloxanes as well as polydialkyl siloxanes including amine and/or hydroxy groups.
Glycosidically-substituted silicones.

Film-forming polymers that are preferably used according to the present invention due to the cosmetic effects thereof in combination with the copolymers a) and b) are, in particular, polyvinylpyrrolidones (INCI designation: PVP) and vinylpyrrolidone/vinyl acetate copolymers (INCI designation: VP/VA Copolymer), wherein the proportion by weight of these polymers is preferably limited to quantities between 1.0 and 10 wt %. Especially preferred cosmetic agents according to the present invention are therefore characterized by further including—relative to the total weight thereof-1.0 to 10 wt % polyvinylpyrrolidone and/or vinylpyrrolidone/vinyl acetate copolymer, preferably polyvinylpyrrolidone. Especially preferred cosmetic agents have a proportion by weight of the polyvinylpyrrolidone and/or vinylpyrrolidone/vinyl acetate copolymer c) to the total weight of the cosmetic agent of 2.0 to 8.5 wt %, preferably 3.0 to 7.0 wt %.

In summary, cosmetic agents that are especially preferred according to the present invention include three mutually different polymers, with the copolymers a) and b) as well as the film-forming polymer c). In a preferred embodiment, the cosmetic agents include, relative to the total weight thereof,
a) 0.1 to 2.0 wt % at least one copolymer a) that is obtained by reacting
   at least one monomer (A1) from the group consisting of acrylic acid, methacrylic acid, C₁-C₆ alkyl acrylic acid esters, and C₁-C₆ alkyl methacrylic acid esters, with
   at least one monomer (A2) from the group consisting of C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates, or C₁₀₋₃₀ alkyl PEG itaconates;
b) 0.2 to 12 wt % at least one polymeric quaternary ammonium compound from the group consisting of vinylpyrrolidone copolymers; and
c) 1.0 to 10 wt % polyvinylpyrrolidone.

Especially preferred are cosmetic agents that include, relative to the total weight thereof,
a) 0.3 to 1.0 wt % a copolymer a) from the group consisting of compounds having the INCI designation Acrylates/C₁₀₋₃₀ Alkyl Methacrylate Crosspolymer;
b) 1.5 to 4.0 wt % a copolymer b) from the group consisting of compounds having the INCI designation Polyquaternium 28; and
c) 3.0 to 7.0 wt % polyvinylpyrrolidone.

Protein hydrolyzates and/or derivatives thereof may be used as a conditioner. Protein hydrolyzates are product mixtures that are obtained by acidically, basically, or enzymatically catalyzed breakdown of proteins. The term “protein hydrolyzates” is also understood according to the present invention to mean total hydrolyzates as well as individual amino acids and derivatives thereof, and mixtures of different amino acids. The molecular weight of the protein hydrolyzates that can be used according to the present invention lies between 75 (the molar weight of glycine) and 200,000; preferably, the molecular weight is 75 to 50,000, very especially preferably 75 to 20,000.

Another group of conditioners is vitamins, provitamins, vitamin precursors, and/or derivatives thereof. Those vitamins, provitamins, and vitamin precursors that are usually assigned to the groups A, B, C, E, F, and H are preferred according to the present invention.

Further conditioners are glycerol, propylene glycol, panthenol, caffeine, nicotinamide, and sorbitol.

Plant extracts may be used as a conditioner, as well as monosaccharides or oligosaccharides and/or lipids.

Preferred cosmetic agents are based on an aqueous or aqueous/alcoholic carrier. The proportion of water to the total weight of the cosmetic agents according to the present invention is preferably at least 10 wt %, in particular, at least 20.0 wt %, further preferably at least 40 wt %, especially preferably at least 70 wt %. The water content of the cosmetic agents according to the present invention is preferably 10 to 98 wt %, preferably 30 to 98 wt %, especially preferably 50 to 98 wt % and, in particular, 70 to 97 wt %.

The compositions of some cosmetic agents that are used can be found in the following tables (all amounts given in wt % are relative to the total weight of the cosmetic agent, unless otherwise indicated).

| | Formula 1 | Formula 2 | Formula 3 | Formula 4 | Formula 5 |
|---|---|---|---|---|---|
| Copolymer | 0.1 to 2.0 | 0.2 to 1.5 | 0.3 to 1.0 | 0.3 to 1.0 | 0.4 to 0.6 |
| a)¹⁾ | | | | | |
| Copolymer | 0.2 to 12 | 0.5 to 10 | 0.5 to 10 | 1.0 to 6.0 | 1.5 to 4.0 |
| b)²⁾ | | | | | |
| Optional | to 100 | to 100 | to 100 | to 100 | to 100 |
| additives | | | | | |

| | | | | | Formula |
|---|---|---|---|---|---|
| | Formula 6 | Formula 7 | Formula 8 | Formula 9 | 10 |
| Copolymer | 0.1 to 2.0 | 0.2 to 1.5 | 0.3 to 1.0 | 0.3 to 1.0 | 0.4 to 0.6 |
| a)¹⁾ | | | | | |
| Copolymer | 0.2 to 12 | 0.5 to 10 | 0.5 to 10 | 1.0 to 6.0 | 1.5 to 4.0 |
| b)²⁾ | | | | | |
| Water | 10 to 98 | 10 to 98 | 30 to 98 | 50 to 98 | 70 to 97 |
| Optional | to 100 | to 100 | to 100 | to 100 | to 100 |
| additives | | | | | |

| | Formula | Formula | Formula | Formula | Formula |
|---|---|---|---|---|---|
| | 11 | 12 | 13 | 14 | 15 |
| Copolymer | 0.1 to 2.0 | 0.2 to 1.5 | 0.3 to 1.0 | 0.3 to 1.0 | 0.4 to 0.6 |
| a)³⁾ | | | | | |
| Copolymer | 0.2 to 12 | 0.5 to 10 | 0.5 to 10 | 1.0 to 6.0 | 1.5 to 4.0 |
| b)²⁾ | | | | | |
| Optional | to 100 | to 100 | to 100 | to 100 | to 100 |
| additives | | | | | |

| | Formula | Formula | Formula | Formula | Formula |
|---|---|---|---|---|---|
| | 16 | 17 | 18 | 19 | 20 |
| Copolymer | 0.1 to 2.0 | 0.2 to 1.5 | 0.3 to 1.0 | 0.3 to 1.0 | 0.4 to 0.6 |
| a)³⁾ | | | | | |
| Copolymer | 0.2 to 12 | 0.5 to 10 | 0.5 to 10 | 1.0 to 6.0 | 1.5 to 4.0 |
| b)²⁾ | | | | | |
| Water | 10 to 98 | 10 to 98 | 30 to 98 | 50 to 98 | 70 to 97 |
| Optional | to 100 | to 100 | to 100 | to 100 | to 100 |
| additives | | | | | |

| | Formula | Formula | Formula | Formula | Formula |
|---|---|---|---|---|---|
| | 21 | 22 | 23 | 24 | 25 |
| Copolymer | 0.1 to 2.0 | 0.2 to 1.5 | 0.3 to 1.0 | 0.3 to 1.0 | 0.4 to 0.6 |
| a)¹⁾ | | | | | |
| Copolymer | 0.2 to 12 | 0.5 to 10 | 0.5 to 10 | 1.0 to 6.0 | 1.5 to 4.0 |
| b)⁴⁾ | | | | | |
| Optional | to 100 | to 100 | to 100 | to 100 | to 100 |
| additives | | | | | |

| | Formula | Formula | Formula | Formula | Formula |
|---|---|---|---|---|---|
| | 26 | 27 | 28 | 29 | 30 |
| Copolymer | 0.1 to 2.0 | 0.2 to 1.5 | 0.3 to 1.0 | 0.3 to 1.0 | 0.4 to 0.6 |
| a)¹⁾ | | | | | |
| Copolymer | 0.2 to 12 | 0.5 to 10 | 0.5 to 10 | 1.0 to 6.0 | 1.5 to 4.0 |
| b)⁴⁾ | | | | | |
| Water | 10 to 98 | 10 to 98 | 30 to 98 | 50 to 98 | 70 to 97 |
| Optional | to 100 | to 100 | to 100 | to 100 | to 100 |
| additives | | | | | |

| | Formula | Formula | Formula | Formula | Formula |
|---|---|---|---|---|---|
| | 31 | 32 | 33 | 34 | 35 |
| Copolymer | 0.1 to 2.0 | 0.2 to 1.5 | 0.3 to 1.0 | 0.3 to 1.0 | 0.4 to 0.6 |
| a)³⁾ | | | | | |
| Copolymer | 0.2 to 12 | 0.5 to 10 | 0.5 to 10 | 1.0 to 6.0 | 1.5 to 4.0 |
| b)⁴⁾ | | | | | |
| Optional | to 100 | to 100 | to 100 | to 100 | to 100 |
| additives | | | | | |

| | Formula | Formula | Formula | Formula | Formula |
|---|---|---|---|---|---|
| | 36 | 37 | 38 | 39 | 40 |
| Copolymer | 0.1 to 2.0 | 0.2 to 1.5 | 0.3 to 1.0 | 0.3 to 1.0 | 0.4 to 0.6 |
| a)³⁾ | | | | | |
| Copolymer | 0.2 to 12 | 0.5 to 10 | 0.5 to 10 | 1.0 to 6.0 | 1.5 to 4.0 |
| b)⁴⁾ | | | | | |
| Water | 10 to 98 | 10 to 98 | 30 to 98 | 50 to 98 | 70 to 97 |
| Optional | to 100 | to 100 | to 100 | to 100 | to 100 |
| additives | | | | | |

| | Formula | | | | |
|---|---|---|---|---|---|
| | 41 | Formula 42 | Formula 43 | Formula 44 | Formula 45 |
| Acrylate/C₁₀₋₃₀ | 0.1 to 2.0 | 0.2 to 1.5 | 0.3 to 1.0 | 0.3 to 1.0 | 0.4 to 0.6 |
| alkyl | | | | | |
| methacrylate | | | | | |
| crosspolymer | | | | | |
| Polyquaternium- | 0.2 to 12 | 0.5 to 10 | 0.5 to 10 | 1.0 to 6.0 | 1.5 to 4.0 |
| 28 | | | | | |
| Optional | to 100 | to 100 | to 100 | to 100 | to 100 |
| additives | | | | | |

| | Formula | | | | |
|---|---|---|---|---|---|
| | 46 | Formula 47 | Formula 48 | Formula 49 | Formula 50 |
| Acrylate/C₁₀₋₃₀ | 0.1 to 2.0 | 0.2 to 1.5 | 0.3 to 1.0 | 0.3 to 1.0 | 0.4 to 0.6 |
| alkyl | | | | | |
| methacrylate | | | | | |
| crosspolymer | | | | | |
| Polyquaternium- | 0.2 to 12 | 0.5 to 10 | 0.5 to 10 | 1.0 to 6.0 | 1.5 to 4.0 |
| 28 | | | | | |
| Water | 10 to 98 | 10 to 98 | 30 to 98 | 50 to 98 | 70 to 97 |
| Optional | to 100 | to 100 | to 100 | to 100 | to 100 |
| additives | | | | | |

| | Formula | Formula | Formula | | |
|---|---|---|---|---|---|
| | 51 | 52 | 53 | Formula 54 | Formula 55 |
| Copolymer a)¹⁾ | 0.1 to 2.0 | 0.2 to 1.5 | 0.3 to 1.0 | 0.3 to 1.0 | 0.4 to 0.6 |
| Copolymer b)²⁾ | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to 100 | to 100 | to 100 | to 100 | to 100 |

| | Formula | Formula | Formula | | |
|---|---|---|---|---|---|
| | 56 | 57 | 58 | Formula 59 | Formula 60 |
| Copolymer a)¹⁾ | 0.1 to 2.0 | 0.2 to 1.5 | 0.3 to 1.0 | 0.3 to 1.0 | 0.4 to 0.6 |
| Copolymer b)²⁾ | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to 100 | to 100 | to 100 | to 100 | to 100 |

| | Formula | Formula | Formula | | |
|---|---|---|---|---|---|
| | 61 | 62 | 63 | Formula 64 | Formula 65 |
| Copolymer a)³⁾ | 0.1 to 2.0 | 0.2 to 1.5 | 0.3 to 1.0 | 0.3 to 1.0 | 0.4 to 0.6 |
| Copolymer b)²⁾ | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to 100 | to 100 | to 100 | to 100 | to 100 |

| | Formula | Formula | Formula | | |
|---|---|---|---|---|---|
| | 66 | 67 | 68 | Formula 69 | Formula 70 |
| Copolymer a)³⁾ | 0.1 to 2.0 | 0.2 to 1.5 | 0.3 to 1.0 | 0.3 to 1.0 | 0.4 to 0.6 |
| Copolymer b)²⁾ | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to 100 | to 100 | to 100 | to 100 | to 100 |

| | Formula | Formula | Formula | | |
|---|---|---|---|---|---|
| | 71 | 72 | 73 | Formula 74 | Formula 75 |
| Copolymer a)¹⁾ | 0.1 to 2.0 | 0.2 to 1.5 | 0.3 to 1.0 | 0.3 to 1.0 | 0.4 to 0.6 |
| Copolymer b)⁴⁾ | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to 100 | to 100 | to 100 | to 100 | to 100 |

| | Formula | Formula | Formula | | |
|---|---|---|---|---|---|
| | 76 | 77 | 78 | Formula 79 | Formula 80 |
| Copolymer a)¹⁾ | 0.1 to 2.0 | 0.2 to 1.5 | 0.3 to 1.0 | 0.3 to 1.0 | 0.4 to 0.6 |
| Copolymer b)⁴⁾ | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to 100 | to 100 | to 100 | to 100 | to 100 |

| | Formula | Formula | Formula | | |
|---|---|---|---|---|---|
| | 81 | 82 | 83 | Formula 84 | Formula 85 |
| Copolymer a)³⁾ | 0.1 to 2.0 | 0.2 to 1.5 | 0.3 to 1.0 | 0.3 to 1.0 | 0.4 to 0.6 |
| Copolymer b)⁴⁾ | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to 100 | to 100 | to 100 | to 100 | to 100 |

| | Formula | Formula | Formula | | |
|---|---|---|---|---|---|
| | 86 | 87 | 88 | Formula 89 | Formula 90 |
| Copolymer a)³⁾ | 0.1 to 2.0 | 0.2 to 1.5 | 0.3 to 1.0 | 0.3 to 1.0 | 0.4 to 0.6 |
| Copolymer b)⁴⁾ | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to 100 | to 100 | to 100 | to 100 | to 100 |

| | Formula | Formula | Formula | | |
|---|---|---|---|---|---|
| | 91 | 92 | 93 | Formula 94 | Formula 95 |
| Acrylate/C₁₀₋₃₀ alkyl | 0.1 to 2.0 | 0.2 to 1.5 | 0.3 to 1.0 | 0.3 to 1.0 | 0.4 to 0.6 |
| methacrylate | | | | | |
| crosspolymer | | | | | |
| Maltodextrin/VP | 0.2 to 12 | 0.5 to 10 | 0.5 to 10 | 1.0 to 6.0 | 1.5 to 4.0 |
| Copolymer | | | | | |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Optional additives | to 100 | to 100 | to 100 | to 100 | to 100 |

| | Formula | Formula | Formula | | |
|---|---|---|---|---|---|
| | 96 | 97 | 98 | Formula 99 | Formula 100 |
| Acrylate/C₁₀₋₃₀ alkyl | 0.1 to 2.0 | 0.2 to 1.5 | 0.3 to 1.0 | 0.3 to 1.0 | 0.4 to 0.6 |
| methacrylate | | | | | |
| crosspolymer | | | | | |
| Polyquaternium-28 | 0.2 to 12 | 0.5 to 10 | 0.5 to 10 | 1.0 to 6.0 | 1.5 to 4.0 |
| Polyvinylpyrrolidone | 1.0 to 10 | 2.0 to 9.0 | 2.5 to 8.5 | 2.0 to 8.0 | 3.0 to 7.0 |
| and/or | | | | | |
| vinylpyrrolidone/vinyl | | | | | |
| acetate copolymer | | | | | |
| Water | 10 to 98 | 10 to 95 | 30 to 95 | 50 to 95 | 70 to 95 |
| Optional additives | to 100 | to 100 | to 100 | to 100 | to 100 |

at least one monomer (a1) from the group consisting of acrylic acid, methacrylic acid, C₁-C₆ alkyl acrylic acid esters, and C₁-C₆ alkyl methacrylic acid esters, with ¹⁾ hydrophobically modified (meth)acrylic acid copolymer²⁾ polymeric quaternary ammonium compound from the group consisting of vinylpyrrolidone copolymers³⁾ Copolymer a) obtained by reacting
at least one monomer (a2) from the group consisting of C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates, or C₁₀₋₃₀ alkyl PEG itaconates ⁴⁾ Copolymer b) obtained by reacting vinylpyrrolidone with methacrylamidopropyltrimethylammonium chloride

Preferred cosmetic agents exist as a hair gel, hair spray, hair foam, or hair wax.

As previously stated, the aforementioned cosmetic agents are characterized by special hair cosmetic properties, in particular, by properties that are advantageous for temporarily deforming hair. A second subject matter of the present invention is therefore the use of an agent according to the present invention to temporarily deform keratin-containing fibers, in particular, human hair.

A third subject matter of the present invention is a method for temporarily deforming keratin-containing fibers, in particular, human hair, with which a cosmetic agent according to the present invention is applied to the keratinous fibers and the keratinous fibers are temporarily fixed in the shape thereof.

The agents, uses, and methods according to the present invention—and some of the preferred embodiments thereof—are characterized by the following points:
1. A cosmetic agent for temporarily deforming keratinous fibers, including—relative to the total weight thereof—
   a) 0.1 to 2.0 wt % at least one hydrophobically modified (meth)acrylic acid copolymer; and
   b) 0.2 to 12 wt % at least one polymeric quaternary ammonium compound from the group of vinylpyrrolidone copolymer.
2. The agent according to point 1, characterized in that the proportion by weight of the copolymer a) to the total weight of the cosmetic agent is 0.2 to 1.5 wt %, preferably 0.3 to 1.0 and, in particular, 0.4 to 0.6 wt %.
3. The agent according to any of the preceding points, characterized in that the copolymer a) is obtained by reacting
   at least one monomer (a1) from the group consisting of acrylic acid, methacrylic acid, C₁-C₆ alkyl acrylic acid esters, and C₁-C₆ alkyl methacrylic acid esters with
   at least one monomer (a2) from the group consisting of C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates, or C₁₀₋₃₀ alkyl PEG itaconates.
4. The agent according to any of the preceding points, characterized in that the copolymer a) is selected from the group of compounds with the INCI designation Acrylates/C₁₀₋₃₀ Alkyl Methacrylate Crosspolymer.
5. The agent according to any of the preceding points, characterized in that the proportion by weight of the copolymer b) to the total weight of the cosmetic agent is 0.5 to 10 wt %, preferably 1.0 to 6.0 wt % and, in particular, 1.5 to 4.0 wt %.
6. The agent according to any of the preceding points, characterized in that the copolymer b) is obtained by reacting vinylpyrrolidone with methacrylamidopropyltrimethylammonium chloride.
7. The agent according to any of the preceding points, characterized in that the copolymer b) is selected from the group of compounds with the INCI designation Polyquaternium 28.
8. The agent according to any of the preceding points, characterized in that the weight ratio of copolymer a) to copolymer b) is 5:1 to 1:5, preferably 3:1 to 1:3 and, in particular, 2:1 to 1:2.
9. The agent according to any of the preceding points, characterized by further including—relative to the total weight thereof—c) 1.0 to 10 wt % polyvinylpyrrolidone and/or vinylpyrrolidone/vinyl acetate copolymer, preferably polyvinylpyrrolidone.
10. The agent according to point 9, characterized in that the proportion by weight of the polyvinylpyrrolidone and/or vinylpyrrolidone/vinyl acetate copolymer c) to the total weight of the cosmetic agent is 2.0 to 8.5 wt %, preferably 3.0 to 7.0 wt %.
11. The agent according to any of the preceding points, characterized in that the cosmetic agent includes—relative to the total weight thereof-0.01 to 0.2 wt %, preferably 0.01 to 0.1 wt % and, in particular, 0.02 to 0.6 wt % an amino alcohol, preferably 2-amino-2-methylpropanol.
12. The agent according to any of the preceding points, characterized in that the cosmetic agent includes—as calculated by the total weight of the agent—at least 10 wt %, preferably at least 20.0 wt %, further preferably at least 40 wt %, also preferably at least 70 wt % water.
13. A cosmetic agent according to any of the preceding points, characterized in that the agent exists as a hair gel, hair spray, hair foam, or hair wax.
14. Use of the agent according to any of points 1 to 13 to temporarily deform keratin-containing fibers, in particular, human hair.
15. A method for temporarily deforming keratin-containing fibers, in particular, human hair, with which the cosmetic agent according to any of points 1 to 13 is applied to the keratinous fibers and the keratinous fibers are temporarily fixed in the shape thereof.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent for temporarily deforming keratinous fibers, comprising, relative to the total weight thereof,
a) 0.1 to 2.0 wt % at least one hydrophobically modified (meth)acrylic acid copolymer; and
b) 0.2 to 12 wt % at least one polymeric quaternary ammonium compound from the group consisting of vinylpyrrolidone copolymers.

2. The agent according to claim 1, wherein the proportion by weight of the copolymer a) to the total weight of the cosmetic agent is 0.2 to 1.5 wt %.

3. The agent according to claim 1, wherein the proportion by weight of the copolymer a) to the total weight of the cosmetic agent is 0.3 to 1.0%.

4. The agent according to claim 1, wherein the proportion by weight of the copolymer a) to the total weight of the cosmetic agent is 0.4 to 0.6 wt %.

5. The agent according to claim 1, wherein the copolymer a) is obtained by reacting
at least one monomer (a1) selected from the group consisting of acrylic acid, methacrylic acid, C₁-C₆ alkyl acrylic acid esters, and C₁-C₆ alkyl methacrylic acid esters; with
at least one monomer (a2) selected from the group consisting of C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates, and C₁₀₋₃₀ alkyl PEG itaconates.

6. The agent according to claim 1, wherein the proportion by weight of the copolymer b) to the total weight of the cosmetic agent is 0.5 to 10 wt %.

7. The agent according to claim 1, wherein the proportion by weight of the copolymer b) to the total weight of the cosmetic agent is 1.0 to 6.0 wt %.

8. The agent according to claim 1, wherein the proportion by weight of the copolymer b) to the total weight of the cosmetic agent is 1.5 to 4.0 wt %.

9. The agent according to claim 1, wherein the copolymer b) is obtained by reacting vinylpyrrolidone with methacrylamidopropyltrimethylammonium chloride.

10. The agent according to claim 1, wherein the weight ratio of copolymer a) to copolymer b) is 5:1 to 1:5.

11. The agent according to claim 1, wherein the weight ratio of copolymer a) to copolymer b) is 3:1 to 1:3.

12. The agent according to claim 1, wherein the weight ratio of copolymer a) to copolymer b) is 2:1 to 1:2.

13. The agent according to claim 1, further comprising, relative to the total weight thereof, c) 1.0 to 10 wt % polyvinylpyrrolidone and/or vinylpyrrolidone/vinyl acetate copolymer.

14. The agent according to claim 13, wherein c) is polyvinylpyrrolidone.

15. The agent according to claim 13, wherein the proportion by weight of the polyvinylpyrrolidone and/or vinylpyrrolidone/vinyl acetate copolymer c) to the total weight of the cosmetic agent is 2.0 to 8.5 wt %.

16. The agent according to claim 13, wherein the proportion by weight of the polyvinylpyrrolidone and/or vinylpyrrolidone/vinyl acetate copolymer c) to the total weight of the cosmetic agent is 3.0 to 7.0 wt %.

17. A method for temporarily deforming keratin-containing fibers, comprising: applying the cosmetic agent of claim 1 to keratinous fibers and shaping the keratinous fibers.
